(19) United States
(12) Patent Application Publication
(10) Pub. No.: US 2020/0187869 A1
(43) Pub. Date: Jun. 18, 2020

(54) SYSTEM FOR PROVIDING REAL TIME WARNINGS BASED ON CONTINUOUS SENSOR SIGNALS

(71) Applicants: NATIONAL SCIENCE AND TECHNOLOGY DEVELOPMENT AGENCY, Pathum Thani (TH); SOFTWARE ISARA ORDINARY PARTNERSHIP, Bangkok (TH)

(72) Inventors: Surapa THIEMJARUS, Pathum Thani (TH); Isara ANANTAVRASILP, Bangkok (TH)

(73) Assignees: NATIONAL SCIENCE AND TECHNOLOGY DEVELOPMENT AGENCY, Pathum Thani (TH); SOFTWARE ISARA ORDINARY PARTNERSHIP, Bangkok (TH)

(21) Appl. No.: 16/606,918

(22) PCT Filed: Apr. 11, 2018
(86) PCT No.: PCT/TH2018/000019

§ 371 Date: Oct. 21, 2019

(30) Foreign Application Priority Data
Apr. 19, 2017 (TH) 1701002141

(51) Int. Cl. A61B 5/00; A61B 5/11; G08B 21/04

(52) U.S. Cl. CPC A61B 5/746; A61B 5/742; A61B 5/7455; G08B 21/0453; A61B 5/112; G08B 21/0446; A61B 5/1117

(57) ABSTRACT

The present disclosure relates to a system for providing real-time warnings based on continuous sensor signals. In one implementation, such a system may include at least one measuring device configured to measure at least one variable related to a user and at least one processor configured to receive the measured at least one variable related to the user for processing and display. The processing may include context recognition and inference for estimating risk. The display may include a real-time warning presented to the user or a caregiver, e.g., using a device associated with the user or caregiver.

### FIELD OF THE INVENTION

The invention relates to system for recording, analyzing risk(s) of accident(s) or need of assistance and providing real-time warning(s) based on continuous sensor signals

### BACKGROUND OF THE INVENTION

The estimation of growth rate of the elderly population by United Nations Organization from 1990 to 2050 indicates that Thailand is confronting with the situation of rapid growth of the elderly population (aged 60 years and more). Thailand will confront with the problem of the elderly population state earlier when compared with other various developed countries. It was predicted that in 2020, Thailand will have the elderly population more than the childhood population due to continuous decrease in reproduction rate and mortality rate. The elderly people considerably demand health services in prevention, treatment and rehabilitation, but the Government can provide only limited services. Health insurance covers only medical treatment, and not the preventive expense or other service fees. Moreover, the growth in proportion of the elderly population per number of working age population has also resulted in the caretaker shortage problem. It is necessary to adopt the body sensor network technology for supporting the elderly care system in Thailand, emphasizing on the development of the device and system for monitoring, detecting, preventing and/or solving health problems that are often found in the elderly, for instance, skid, fall, bedsore, loss due to dementia or insomnia.

Falling is a fatal accident if happens to the elderly. Examples of other contingent serious dangers are cuts on head, skin abrasion, bone fracture, joint distraction, etc., which entirely affect the daily living of the elderly. For patients in hospitals, falling may occur after the patient has undergone a surgical operation and tried to get up by themselves upon convalescence due to their understanding that they can walk by themselves, or the patients who are dosed for medicines with side effect of stupor, confusion or muscular hypotonia. For these reasons, the effective fall detector is essential. If the device can detect falling and send a need of assistance signal promptly, it will reduce the contingent danger from the fall of the patients and elderlies. The first model of fall detector was developed since the beginning 1970. During that period, the device was only capable of simply sending a remote alert when the user presses the button. Until 1990, the study on automatic fall measurement was conducted. The first prototype of fall measuring device was developed by William et al., using piezoelectric shock sensor and tilt switch in the measurement. At present, fall detectors have been continuously researched and developed, and the number of the elderlies is increasing continuously increased every year, resulting in more attention on fall detectors.

Various forms of fall monitoring and detecting technology have been developed. Most of them are in research level to prototype devices that have been sold in foreign countries. There are several methods for fall behavior detection process subject to the devices used, for example, the use of shock sensor to detect falling shocks, together with the use of mercury tilt switch to detect fall direction. The use of video cameras for fall detection, despite good results with some forms of fall, results in lack of privacy for the patients or elderlies, and has some limitations on lighting condition and field of view constraint. As same as to wear the accelerometer on the wrist or wear on neck like jewelry, although it provides more comfortable feeling, the detection accuracy is relatively low compared to wearing a sensor on waist or chest.

Apart from device that measures physical parameters of the wearer, for example, accelerometer and gyroscope, and devices for measuring the wearer's biological parameters, such as, electrocardiogram (ECG), noninvasive blood pressure (NIBP), pulse oximetry (SpO2), and a body surface temperature (BST), can be used to support fall detection. The use of the device in the latter group requires mutual analysis with an expert physician.

The examples of developed fall detectors, prevention and monitoring devices, and motion sensors are as follows:

1. US Patent Application No. US20080129518 A1 on “Method and system for fall detection” uses a tri-axial accelerometer and impact detector, which may be performed using accelerometer or impact noise measured from body-linked microphone. For fall detection, the device can be worn on the wrist or mounted on the chest. Fall detection shall rely on analysis of acceleration signals in 3 periods, namely, pre-fall period, during fall period, and post-fall period. If the values of acceleration signal at any position and signal value retention duration are within threshold, it indicates that a fall has occurred.

2. US Patent Application No. WO2008129452 A1 on “Multi-sensory fall detection system” is the invention on guideline for fall detection using at least two detectors. Such detectors may be an accelerometer or a vibration sensor, etc. The installation position of detectors may be on the waist, ankle joint or wrist. In the case where detectors are worn on the waist and ankle joint, a fall can be detected by verification of data measured from the detectors at both positions.

3. US Patent Application No. US2009076419 A1 on “ Loss-of-balance and fall detection system” is the use of foot force sensor worn on the joint ankle or shoes together with accelerometer and gyroscope worn on the chest and both sides of the thigh for fall detection. The device on the chest is used distinguish bending, twisting and turning, while device on the thigh is used to distinguish between standing and sitting.

4. US Patent Application No. U.S. Pat. No. 7,714,728 B2 on “Using RFID to prevent or detect falls, wandering, bed egress and medication errors” adopts RFID device for fall detection. RFID tag may be attached on the wrist, ankle, or sock and the signal receiver is mounted on the floor, door, bed side or bed. The system sends out warning upon the detection that RFID tag is approaching the floor.

5. US Patent Application No. US20090292227 A1 on “Fall detection utilizing a three-axis accelerometer” is fall detection based on the degree between the body and the gravity force and the sum of the acceleration signals. The acceleration magnitude is calculated from the tri-axial accelerometer attached to the chest.

6. US Patent Application No. US20110230791 A1 on “ Fall detection and/or prevention system” is the fall detection system based on the tri-axial accelerometer attached to the waist. Falling is detected based on a feature set, such as acceleration, and acceleration signal magnitude, etc. If the value is higher than a predefined value, it is possible that a fall has occurred. If the system detects that a fall occurs, the wearer can respond the circumstance by pressing the button. If the button is not pressed after fall, the system will send out the need of assistance signal. Or if the wearer presses the button, the system will not send signal and will record value derived from the signal for use in examining wrong prediction of a fall accordingly. However, the wearer can press the button to request for assistance without falling.

7. US Patent Application No. US20140313036 A1 on “Fall detection system and method” presents a fall detection system that can adjust algorithm in fall detection. The detection device may be a tri-axial accelerometer and may be mounted on the wrist, torso or neck. Approximately 1.3 second from the device shall be recorded and processed on cloud. Fall detection shall be considered from acceleration signal values in three periods, namely, pre-fall period, during fall period and post-fall period. Normally, the acceleration signal in pre-fall period shall be in the range of 0-0.6 g for around 0.4 second. During fall is the period where the acceleration signal is more than 1.25 g for around 0.3 second, and post-fall is the period where the acceleration signal is close to 1 g for around 0.6 second. If the signal values in three periods are corresponding to the said conditions, fall warning shall be warned by the system.

8. US Patent Application No. U.S. Pat. No. 9,005,141 B1 on “Ambulatory system for measuring and monitoring physical activity and risk of falling and for automatic fall detection” presents monitoring system with accelerometer mounted on the area of chest (upper body) consisting of four modules such as postural transition, gait analysis, assessment on risk of falling and automatic fall detection. The postural transition detection module uses angle between body and gravitational force to classify postural transition such as sitting-standing, standing-sitting, sitting-lying down, etc. Gait analysis is performed based on tri-axial acceleration signals, for example, stepping on the left foot or right foot by analyzing the acceleration signal on the lateral axis, stepping on toe or heel by analyzing the signals on the frontal axis and the vertical axis, and pace speed, etc. Risk of falling are assessed from three values measured during the postural transition, i.e., mean of transition duration, standard deviation of transition duration, and successive transition. All the three values are high in the people who have a fall history. Automatic fall detection is performed by thresholding on the norm of acceleration in frontal and lateral planes. Posture and gesture of the device wearer prior to impact is used for fall confirmation. If the signal which is higher than a threshold or peak occurs after walking or turning posture and followed by any postural transition into lying down posture, the system shall consider different values, such as, width of peak interval signal, speed in vertical axial signal before peak, sum of all three tri-axial acceleration signals at impact time, sum of acceleration in frontal and lateral plane at the time of impact, speed at each axis at the time of impact, and energy of the sum of acceleration in frontal and lateral plane at the time of impact. If the values correspond to the determined conditions, the system shall notify that that fall occurs. Or if the peak does not occur after walking or turning posture, the system shall notify that it is a fall if the peak occurs after a posture that is followed by sitting or lying down posture.

9. US Patent Application No. US20110230791 A1 on “Fall detection and/or prevention systems” presents a sensor belt for fall detection. The user can cancel erroneous warning by the pressing button at the belt.

10. US Patent Application No. US20060001545 A1 on “Non-Intrusive Fall Protection Device, System and Method” presents the procedure of fall impact reduction using cushion inflatable based on signals from several sensors installed at the floor or wall. Cushion at the point where impact occurs is expected to be inflated to support fall, resulting in decrease in fall severity.

11. US Patent Application No. US20050067816 B2 on “Method and apparatus for body impact protection” presents the installation of air bags in clothing to reduce impact of falling based on the analysis of motion signals from sensors installed at the trousers. The system stores user's normal movement data and measures the fall by comparing the signals with fall data and the normal data previously stored.

12. US Patent Application No. US 20070159332 A1 on “Using RFID to prevent or detect falls, wandering, bed egress and medication errors” presents the use of RFID tag to track patients for location. Patients will wear a RFID tag and RFID antennas shall be installed in the areas where patients live, such as on the bed or floor. The system detects movement such as falling, walking, and getting out of bed or room. Falling is detected when the tag on the patient's upper body is near the floor antenna over a specific period of time. Getting out of bed is detected by the tags on the lower body of the user moving away from the bed antenna. When interested information is detected, the system shall instruct the camera to function and transmit the alert to the caretaker. Caregivers will also wear RFID tags so they can be monitored and tracked

13. US Patent Application No. U.S. Pat. No. 8,260,570 B2 on “Method and system for fall-onset detection” presents fall-onset monitoring systems using acceleration and direction sensors located on many parts of the body (such as the hips and waist). Measurements can be performed by comparing sensor signal values with pre-specified values. The comparison shall be performed using rules, which will compare the values in subsequent order. Accelerometer and gyroscope shall be installed on a microcontroller that functions as a processor. When falling occurs, signal light is turned on. This microcontroller can be connected to the network in the future.

14. US Patent Application No. US 20080186189 A1 on “System and method for predicting fall risk for a resident” presents the designed system for predicting fall risk inside the residential area of any resident using data from at least one sensor which is installed in the residential area (such as motion sensor, light sensor, air pressure sensor, and blood pressure sensor, etc.). Fall risk is predictable from gait (such as pace speed, pace length, motion speed, balance, etc.), environmental elements (such as light, surface, barrier, etc.), internal elements (such as intellectual disorder, visually handicap, etc.), and personal data (such as fall history, medication, underlying disease, etc.). The risk prediction can be performed remotely. The models advised for use in fall risk prediction are Bayesian Networks and HMM. The reports are transmitted to the caretaker's devices such as computer, PDA or fax, within a predefined period of time (such as 10 minutes).

15. US Patent Application No. US 20130023798 A1 on “Method for body-worn sensor based prospective evaluation of falls risk in community-dwelling elderly adults” presents the methodology and system for building models for fall risk assessment and finding the best parameters of the model generated from the sensor data. The system is intended for use during timed up and go (TUG) testing. Parameter values such as step length, gait speed and testing time, are calculated and forwarded to feature selection and used for model construction. Wireless sensor is attached on the user and sends signals to computer for feature calculation and risk assessment.

16. US Patent No. U.S. Pat. No. 8,081,082 B2 on “Monitoring patterns of motion” presents a method for monitoring motion patterns by comparing signals from sensor attached to the patient body and the baseline signals of the user earlier stored. If the signal of the corresponding sequence exceeds the set value, it sends a signal to the caretaker. The baseline signal of any bodily movements are created from a collection of data obtained from that patient for several rounds.

17. US Patent Application No. US 20130303860 A1 on “The invention provides methods and systems for assessing fall risk to prevent or lower the incident of patient falls.” presents a system comprising sensor, gateway, client application and web application. The sensor that measures patient movement sends information to the client application and the said data may be transmitted to central web application. Web application can receive data from more than one client application program. Patient databases and records are stored at the central web application. Sensors herein may refer to scales, accelerometer, video image, or sensors that give location, force, motion rate, or physical outcome of motion by the musculoskeletal system. The user of client application program can assess fall risk with defined testing protocol and evaluate the potential risks. This can be compared with the testing results of other patients.

According to the aforesaid inventions, it was deemed that most inventions are technologies related to fall detection, fall risk assessment or fall prevention using inflatable mounting for impact abatement. It does not cover continual analysis of risk information and real-time warning when the measured person is at risk of accident(s) or requires aid prior to the actual incident.

### SUMMARY OF INVENTION

This invention relates to a system for recording, analyzing risk(s) of accident(s) or need of assistance and providing real-time warning(s) based on continuous sensor signals, where in the system according to this invention comprising at least one measuring device and at least one administrative device. Wherein said measuring device receives various data related to an examinee to process and display results to an administrative device. More complex system may comprise at least one signal fusion device for transferring data related to the examinee (such as age of patient or elderly) for a wider area of coverage, and/or at least one server that records various data related to the examinee and an administrator, in order to playback, process and/or transfer data to other administrative device(s) or transfer warning data to relative(s) of the examinee. The data analytical-processing step may be performed on the measuring device(s) itself or distributed to various parts of the system. Wherein said data processing step comprises a data processing and displaying step, which further comprising a data input step, a context recognition step, an inference for estimate risk and a warning step, a database record step, a display step, and a warning determine step.

In eastern counties including Thailand, there are considerable families characterized as extended family and/or hiring a caretaker for a patient or an elder. A warning when the patient or the elder already fell or had an accident is less useful than a monitoring to prevent falling or accident before it happens. It also provides support in the general daily routine such as but not limited to going to the toilet, walking or getting out of bed. The system according to the invention is used to continuously measure the context data of the person who is measured to continually record, analyze, evaluate the risk, and perform a real-time warning when they are in an incident(s) or any bodily movement occurred that may pose a potential risk (including but not limited to falls, bedsore, loss, etc.) to allow the caretaker to correct the situation promptly before such adverse event occurs. The benefits of the use of continuous careful monitoring in this way are to prevent any danger and occurrence of the adverse event for the person who is measured, to reduce the burden on caretakers. So, the caretakers can do other tasks while elderlies or patients is at low risk (e.g., sleeping, etc.), and do not need to stay alert for them all the time. The caretakers or relatives of the examinees can use this system to alert the caretakers to take care them at the designated time (e.g., overturn or exercise), or to assess the caretakers whether they perform the task at the scheduled time. In addition, the physician or caretaker can use this data to more accurately analyze users' daily routines than interviewing from users or their relative(s).

### BRIEF DESCRIPTION OF DRAWINGS

FIG. 1 shows a system component for recording, analyzing risk(s) data based on continuous sensor signals and providing real-time warning(s).

FIG. 2 shows a system component type 1, comprising at least one measuring device and administrative device(s).

FIG. 3 shows a system component type 2, comprising at least one measuring device, administrative device(s), and server(s).

FIG. 4 shows a system component type 3, comprising at least one measuring device, signal fusion device(s), and server(s).

FIG. 5 shows a system component type 4, comprising at least one measuring device, signal fusion device, and server(s) that the data has been transferred to server(s) through administrative device(s).

FIG. 6 shows a system component type 4, comprising at least one measuring device and signal fusion device and server(s) that data has been transferred to server through signal fusion device(s).

FIG. 7 shows a screenshot of some display used in nursing home.

### DETAILED DESCRIPTION OF THE INVENTION

The foregoing detailed description of preferred embodiments of the invention will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

The system for recording, analyzing risk(s) of accident(s) or need of assistance and providing real-time warning(s) based on continuous sensor signals comprising:
at least one measuring device (11) which receive(s) various data related to the user(s) or the examinee(s),
at least one administrative device (12) which receives the data related to the user or the examinees from the measuring device(s) (11) for displaying to the administrator.

Wherein the processing and displaying of the data occurred at the measuring device(s) (11) and/or the administrative device(s) (12), in which the system having continual analysis for risk of accident(s) or need of assistance based on the data received from the measuring device(s) or in a combination of with any one of an additional data on profile of examinee, location of examinee, or time, and can provide warning(s) prior to an occurrence of unwanted incident(s).

In another embodiment of the system for recording, analyzing risk(s) of accident(s) or need of assistance and providing real-time warning(s) based on continuous sensor signals comprising:
at least one measuring device (11) which receive(s) various data related to the user(s) or the examinee(s),
at least one administrative device (12) which receives the data related to the user or the examinees from the measuring device(s) (11) for displaying to the administrator, and
at least one server (13), which records various data related to the user or the examinee, and administrator, in order to allow playback, and/or process, and/or transfer of the data between the administrative device(s) (12).

Wherein the processing and displaying of the data occurred at the measuring device(s) (11), and/or the administrative device(s) (12), and/or the server(s) (13), in which the system having continual analysis for risk of accident(s) or need of assistance based on the data receiving from the measuring device(s) or in a combination of with any one of the additional data on profile of examinee, location of examinee, or time, and can provide warning(s) prior to an occurrence of unwanted incident(s).

In another embodiment of the system for recording, analyzing risk(s) of accident(s) or need of assistance and providing real-time warning(s) based on continuous sensor signals comprising:
at least one measuring device (11) which receive(s) various data related to the user(s) or the examinee(s),
at least one signal fusion device (14), which receives the signal or the data from measuring device(s) (11) and transfers the data to the administrative device(s) (12), and
at least one administrative device (12), which receives the data related to the user or the examinees from the signal fusion device(s) (14) for displaying to administrator.

Wherein the processing and displaying of the data occurred at the measuring device(s) (11), and/or signal fusion device(s) (14), and/or administrative device(s) (12), in which the system having continual analysis for risk of accident(s) or need of assistance based on data receiving from the measuring device(s) or in a combination of with any one of the additional data on profile of examinee, location of examinee or time and can give warning prior to an occurrence of an unwanted incident.

And in another embodiment of the system for recording, analyzing risk(s) of accident(s) or need of assistance and providing real-time warning(s) based on continuous sensor signals comprising:
at least one measuring device (11), which receive(s) various data related to the user(s) or the examinee(s),
at least one signal fusion device (14), which receives the signal or the data from measuring device(s) (11) and transfers the data to administrative device(s) (12),
at least one administrative device (12), receive(s) data related to the user(s) or the examinee(s) from the signal fusion device(s) (14) for displaying to administrator(s), and
at least one of the server(s) (13), which record(s) various data related to the user(s) and the administrator(s), in order to allow playback, and/or process, and/or transfer of the data between the administrative device(s) (12) or receives the data related to the user(s) or the examinee(s) from the signal fusion device(s) (14) before transferring the data between administrative device(s) (12)

Wherein the processing and displaying of the data occurred at the measuring device(s) (11), and/or signal fusion device(s) (14), and/or administrative device(s) (12), in which the system having continual analysis for risk of accident(s) or need of assistance based on data receiving from the measuring device(s) or in a combination of with any one of the additional data on profile of examinee, location of examinee or time and can give warning prior to an occurrence of an unwanted incident.

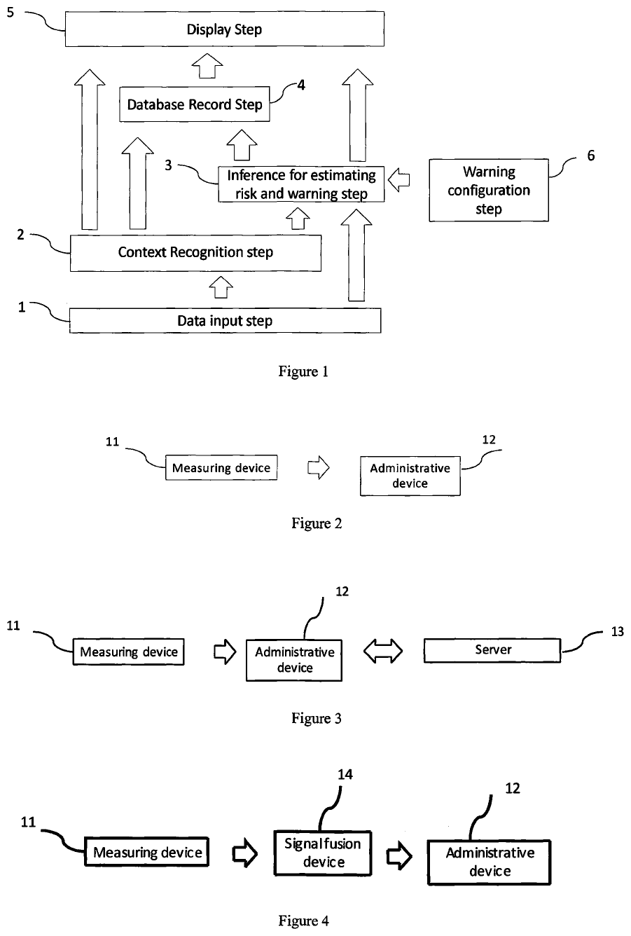

Moreover, the system for recording, analyzing risk(s) of accident(s) or need of assistance and providing real-time warning(s) based on continuous sensor signals in every embodiment as mentioned in this invention contains the ability to analyze continuous data and provides instant warning(s) when detecting whether the examinee is at risk or need assistance, in order to allow the administrator to prevent the unwanted incident(s) or can resolve the situation in time. In which the system contains step of processing and displaying the data which has the coherence as show in FIG. 1 as follows:
Data input step (1) measures various data related to the user(s) or the examinee(s) continuously from measuring device(s) (11) or various data related to the device itself.
Context recognition step (2) converts the signal(s) received from the data input step (1) and transfers to the database record step (4), and/or transfers to the inference for estimating risk and warning step (3), and/or transfers to the display step (5).
Inference for estimating risk and warning step (3) receives the data from the data input step (1) and/or the context recognition step (2), and matches the measured data, and/or the context data, and/or at least one quantitative metric calculated from data obtained from the data input step (1), and/or the context data input step (2) with warning rule(s) which configured in warning configuration step (6) to calculate whether the warning(s) should be warned and transfers the result(s) to the display step (5) and/or records the obtained result(s) by the database record step (4).
Database record step (4) records various parameters received from the context recognition step (2) and/or the inference for estimating risk and warning step (3) for future use.
Display step (5) immediately illustrates the data received from the context recognition step (2) and/or the inference for estimating risk and warning step (3) in form of instant warning(s) or illustrates the data which recorded in the database by the database record step (4) for using in analysis and/or using the data for making further decision.
Warning configuration step (6) wherein warning rule(s) are specified in the system and/or selected by the administrator(s) from the occurred incident(s) that requires warning(s) to be sent to the administrator(s) or assigned related person(s). The characteristic(s) of the warning(s) will be predetermined in the system or be configured by the administrator(s). In which the warning rule(s) is determined using a combination of either one or more of the risk factor(s) of the user(s), or operation period of the system, or characteristics of symptom(s) of the user(s), or the user(s)'s context, or characteristics of device(s), or characteristics of received or acquired signal(s), or quantity of received or acquired signal(s), or characteristics of location of the user(s), or sound frequency, or location of the user(s), or heart rate of the user(s), or temperature, or pressure, or humidity.
   Wherein details of the data processing and displaying step in FIG. 1 are as follows:

Data input step (1) continuously receives various data from measuring device(s) (11), such as, movement, heart rate, temperature, pressure, sound, photo, and video image, etc., or various data related to the device itself, such as, contact, location, signal strength, battery strength, etc., in which may be installed on small size electronic device attached to the user in the form of wearable, or implantable, and/or may be the user's data measuring device(s) that has been installed to ambient environment.

Context recognition step (2) converts the occurred signal from the user's data measuring device(s) (11), such as:
Converts the data from image signal(s), wearable movement sensor signal(s), or motion capture system into various contexts, such as activity (e.g., stand, sit, sleep, walk, run, jump, stand up, walk up or down stair, various sleep positions), various exercise postures, movement of any part of the body, including processing of movement data into quantitative metrics including but not limited to the number of step(s) or energy used from gesture recognition or activity recognition process(es).
Converts the data from microphone array to direction of sound from sound source localization for example, work of (Teachasrisaksakul K, Thiemjarus S, Polprasert C. Speaker tracking module for indoor robot navigation. In: Proceedings of ECTI-CON, Hua-Hin, Thailand, 2012.)
Converts the data from microphone to sound type(s), such as, crying sound, speaking sound, applauding sound, sound from impact of objects within the room, from sound analysis procedure.
Converts the data of wireless signal(s) to user's coordinates, user's room, or evaluates whether the user has stayed away from or passed the designated point.
Converts the data from ambient sensor(s), such as temperature sensor, force sensor, distance sensor, including RFID device(s), to location of user in the room or contact with object(s) in the room.
Converts the data of vital sign(s) to various emotions, for example, work of (Picard R W, Healey J. Affective wearables. In: Proceedings of the First International Symposium on Wearable Computers 1997; 90-97.)
Converts data from touch sensor(s) to the state of device whether it is still attached to the body of user or detached/removed etc.
Converts battery voltage to the state of device whether the battery is full or low, etc.
   Wherein the processing may be on the circuit or any device or distributed on one or more device(s), which has data fusion process at one or several point(s) through the network system. In which the processing provides more accuracy, speed, and/or energy efficiency, and/or allow to rapidly transfer of the data between the device(s). Wherein the method for distributed analysis of context data will consider various factors, such as the measured data from the device(s) in the system, processing capacity (such as, memory size, processor speed, etc.), power source, and limitations of the data transfer channel(s) (for example, iBeacons has the space of 20 of 27 bytes for transferring user's data). Examples are:
From system testing, when using button type battery, model CR1616, which has the capacity about 50-55 milliampere per hour (mAh), the miniaturized tri-axial acceleration measuring device can continuously transfer raw signals sampled at 100 hertz frequency to the mobile device up to 6 hours and up to 2 months if transfer only Bluetooth low energy beacon signal. In a developed system which the context recognition step 2) is processed on the device(s) instead of transferring the raw data to process directly in administrative device(s) (12), with the result(s) acquired from signal analysis transferred via beacon signal, can prolong the usage time, in which the said duration will depend on the algorithm and frequency of signal transmission.
Motion sensor device A, which is attached on the leg, transfers mean data and variance data of the signals to mobile phone, and motion sensor device B, which is attached on the body, transfers mean data and variance data of the signals to mobile phone. The data fusion, for converting to context data, may occur on the mobile phone before the data is transferred to the inference for estimating risk and warning step (3), the database record step (4), the display step (6), or is transferred to other device(s) for data fusion.
Motion sensor device A, which is attached on the wrist, performs context recognition based on three dimensional acceleration signals and transfers the acquired results via iBeacon to administrative device(s) (12) for every half second (the data of falling represents by number 1 and 0, motion data represents by the number (such as supine=1, lie on the left=2, prone=3, etc.), motion data represents number 1 and 0 and battery strength). Motion sensor device B, which is attached on the waist, performs context recognition based on three dimensional acceleration signals and transfers the acquired result via iBeacon. Due to the movement occurred only on the wrist, motion data from motion sensor device A has value of 1 and the motion data from motion sensor device B has value of 0. At the administrative device(s) (12), the motion data will be integrated and determined as 1 as movement of any part of the body is considered as body movement. To determine activity, majority vote can be used, or in the case where majority vote does not work, the results can be derived from the sensor device(s) that is more accurate, etc.
Inference for estimating risk and warning step (3) in which the technique of this step may be in the form of rule-based system, expert system, programming by using branching structure (such as applying if . . . else or switch . . . case in the program), and/or inference mechanism (such as integrated system between rule-based system and time-controlled system in the program), and/or any other method(s) that has not been mentioned herein.
Database record step (4) records various parameters acquired from the context recognition step (2) and/or the inference for estimating risk and warning step (3) in the form of file and folder, relational database, non-relational database, object-oriented database, temporal database, etc. for future use.
Display step (5) refers to displaying of warning(s) or data visualization in various forms such as table(s), chart(s), graph(s), for analysis and assessment for making further decision, for making summary report and/or for evaluating both the examinee (such as activity daily living and daily sleeping patterns) and administrator (such as time period between the incident(s) and intervention(s), forgetting to turn over the patient or not, etc.). The instant warning(s) can be in the form of sound, and/or light, and/or vibration, and/or monitor display at the measuring device(s) (11) and/or the signal fusion device(s) (14) (such as hub, router, microcontroller, other measuring device(s), other administrative device(s), other computer(s), etc.), and/or administrative device(s) (12) (such as mobile phone, tablet, smart watch, other displaying electronic device(s), etc.), including the data transmission via other network(s) in the form of SMS, electronic mail, chat, and/or contacting via telephone to administrator, call center or relatives.
Warning configuration step (6) is the selection of incident(s) in which when occurred, warning(s) should be sent to administrator(s) and/or related person(s) as configured in the system. The warning(s) can occur at least at one point in the system, in which the characteristic of the warning(s) may be the default values or configured in the system by the administrator. The warning rule(s) determines signal data, and/or user context(s), and/or device context(s), along with the relationship(s) of said data with place, time, and/or quantitative value(s) of the signal(s), etc., for identifying the incident(s) that triggers warning(s).

Examples of warning rules are:

1. Warning when the examinee moves (motion→motion_warning)

2. Warning when the examinee's movement is over a threshold (level(motion)>3→motion_warning)

3. Warning when the examinee stands up (stand→stand_warning)

4. Warning when the examinee walks or runs (walking OR running→movement_warning)

5. Warning when the examinee does not move over a predefined period (no_motion>time_duration→no_motion_warning)

6. Warning when the examinee does not change the sleep posture in daytime over a predefined period (lying AND no_posture_change>time_duration AND day→no_posture_change_warning)

7. Warning when the examinee falls (fall→fall_warn)

8. Warning when the examinee walks during the night (walking AND night→walk_at_night_warning)

9. Warning when the examinee goes to bathroom at night (bathroom AND night→bathroom_at_night_warning)

10. Warning when the examinee goes up or down stair at night ((walk_upstairs OR walk_downstairs) AND night→stair_warning)

11. Warning when there is no signal from the examinee in the prespecified area(s) sent to receiver (e.g. not(bedroom) AND not(living_room) AND not(bathroom)→warning (Assume there are 3 rooms)

12. Warning when the examinee goes outside the house (e.g. outside(GPS) AND not(bedroom) AND not(living_room) AND not(bathroom)→warning (GPS is outside and the signal fusion devices (14) in the house do not receive any signal from the measuring device (11))

13. Warning when battery is low (level(battery)<1.5→battery_warning)

14. Warning when the sensor device is detached (e.g. not(skin_sensor)→sensor_off_warning)

Wherein the administrator can add other rule(s) in addition to the mentioned above.

The default warning configuration can be specified by the developer or the administrator is allowed to select the specific warning topic(s) for a specific examinee. For example, rule number 6 is used for patient that has a risk of pressure ulcers to warn the administrator to turn the body of the patient periodically. By recording sleep postures of the patient into the database, whether the administrator has turned the body of the patient according to the schedule can be checked. Wherein rule number 1 applies for patient who requires special care (such as ICU or post-operative patient). The nurse must pay extra attention when the patient is trying to leave bed in order to prevent falling, etc. The complexity of configuring, alerts and notification methods can be changed according to the user. For example, in general, the administrator (caretaker) in a hospital may want to have an icon to notify that a patient is in motion, however, for patients that need special care, the administrator may require a special configuration for both sound and icon notifications. In the case that the system supports more than one users and the administrator(s) uses more than one measuring device(s) (11) for an examinee, the administrator can make a personalized configuration for warning(s) or measurement for each examinee. This can be done by assigning each measuring device(s) (11) or each group of measuring device(s) (11) to the examinee, along with the warning configuration for the said (group) measuring device(s) (11). The default warning configuration from the system can also be used.

The administrator can configure the warning(s) and/or use at least one measuring device(s) (11), of the same or different type(s), for each examinee.

In which the said system also specifies the location of the examinee inside and/or outside the building along with the warning(s). Moreover, warning(s) can be triggered by the occurrence of any one of or in a combination of standing up, or body movement, or no body movement for a specified period, or leaving the specified area, or no change in sleep posture for a specified period, or walking up or down stairs, or getting up, or walking at night, or going to bathroom, or going to bathroom at night, or going outside the house, or falling, or walking.

Moreover, the warning(s) can be triggered by sensor detachment, low battery, and/or connection problem. The system can display playback history of examinee context and warning(s) a, and/or display playback warning(s), or display the examinee context and/or location immediately, and can identify the signal fusion device(s) (14) that is malfunctioned. In which the system allows personalized warning configuration to be specified for each examinee.

Each step, which is the composition of the system for recording, analyzing risk(s) of accident(s) or need of assistance and providing real-time warning(s) based on continuous sensor signals according to this invention, can be distributed to various parts of the system which may contain different composition in order to be suitable for various situation(s) as following examples

System type 1 according to FIG. 2 comprising at least one measuring device(s) (11) and administrative device(s) (12) such as computer, tablet, mobile phone, smart watch, pager, monitor, or other displaying devices, etc. The system in this manner is suitable for the case that the examinee is near the administrator such as home care, or in ICU, etc. In the case of more than one measuring device(s) (11), the device may be the same type or different types and may be on the same examinee or many examinees (for monitoring a number of patients/elderlies in the same time).

System type 2 according to FIG. 3 comprising at least one measuring device(s) (11), administrative device(s) (12), and server(s) (13), which records various data for playback or transferring to other administrative device(s) (12). The central server may refer to a local server, a remote server, or a group of computers located in the network, such as, a cloud server, etc. The system in this manner is suitable for the case that the examinee is near the administrator, but requires recording and/or allows other devices to access any part of the data.

System type 3 according to FIG. 4 comprising at least one measuring device(s) (11), signal fusion device(s) (14), and administrative device(s) (12). The system in this manner is suitable for the case that the administrator is too far to directly receive the signal(s) from the measuring device(s) (1), such as in another room or in different floors. By having more than one signal fusion device(s) (14) in different locations, it is able to identify the location of the examinee from the location of the signal fusion device(s) that receives the signal(s) from measuring device(s) (11). In which the measuring device(s) (11) can connect and transfer the data through new signal fusion device (14) instead of connecting to the former signal fusion device(s) (14) when moving from the location of the former signal fusion device(s) (14) to the location of the new signal fusion device(s) (14).

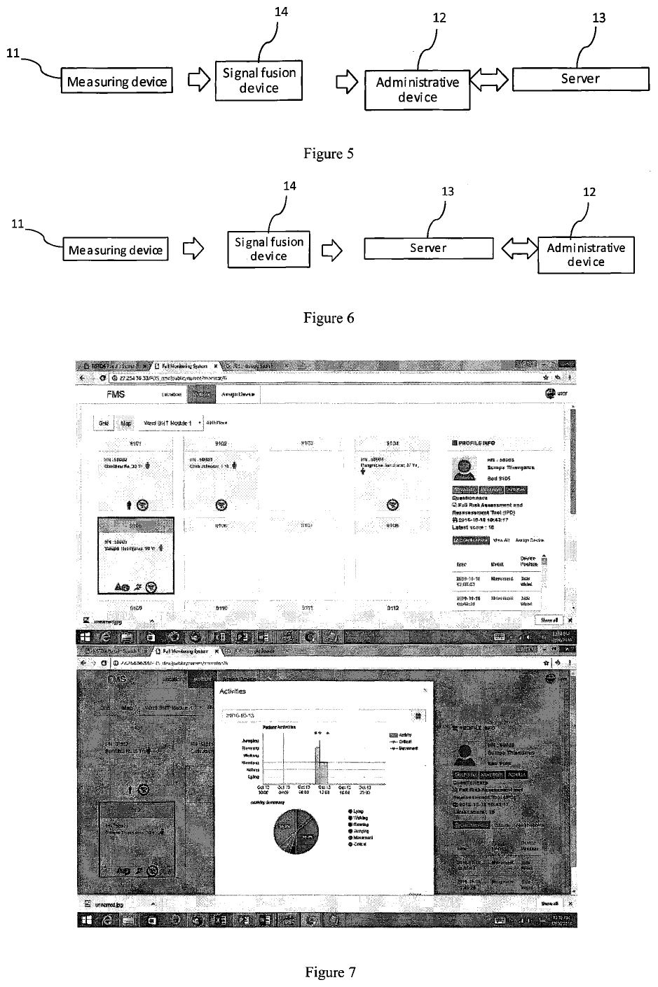

System type 4 comprising at least one measuring device(s) (11), at least one signal fusion device (14), administrative device(s) (12), and server(s) (13), in which the usage is similar to the system type 3, but having the data recording in order to playback or transfer the data to other administrative device(s) (12). Wherein the data will be transferred to the server(s) (13) through the administrative device(s) (12) (as shown in FIG. 5) or though one or many signal fusion device(s) (14) (as shown in FIG. 6), or both types of devices. Wherein any administrative device(s) (12) can receive the data from either or both of the server(s) (13) or the signal fusion device(s) (14) for displaying. The system that contains data transmission according to FIG. 5 is suitable for using in healthcare centers. An example screenshot is shown in FIG. 7. Server(s) (13) may be used for separating results for displaying on one or more administrative device(s) (12) or the display can also be made on the server(s) (13).

Moreover, the system for recording, analyzing risk(s) of accident(s) or need of assistance and providing real-time warning(s) based on continuous sensor signals according to this invention further comprising a basic examinee evaluation step by using questionnaire(s), and/or other test(s), and/or score(s) from the test(s) of examinee. Wherein the administrator will fill the form for evaluating fundamental risk(s) or level of the severity of illness and decide for the suitable measuring device(s) and warning parameter(s). Wherein the said evaluation will be implemented at specified interval (such as every 8 hours or every nurse working shift). The data, such as age, medication that results in stupor, post-operative examinee, or examinee that fall within one month, will be evaluated in order to determine initial risk and the suitable configuration of continuous measurement.

Based on the development of algorithms for converting the data from motion sensor device(s) to various contexts in order to be used with the system for recording, analyzing risk(s) of accident(s) or need of assistance and providing real-time warning(s) based on continuous sensor signals, it is found that the motion measurement by placing sensor device on different parts of the body will provide different recognition accuracy. The test in more than 10 subjects provide the recognition accuracy as summarized in following table:

| | | Front | Lateral | | Upper | | Upper | |
|---|---|---|---|---|---|---|---|---|
| | Chest | Hip | Hip | Ear | Arm | Wrist | Leg | Ankle |
| Falling | 84.62 | 86.15 | 91.15 | 86.92 | 78.08 | 86.54 | 85.38 | 81.92 |
| Standing Up | 98.18 | 100 | — | — | — | 96.36 | — | 94.55 |
| Sleep | 99.9 | 99.81 | 99.92 | 99.47 | 99.84 | 88.48 | 98.1 | 99.98 |
| Basic Activity | 97.2 | 97.95 | 97.65 | 87.73 | 97.06 | 94.4 | 98.47 | 98.37 |

### BEST MODE

As mentioned in detailed description of the invention.

1-17. (canceled)

18. A system for providing real-time warnings based on sensor signals, comprising:
at least one sensor configured to detect at least one variable related to a user; and
at least one processor configured to:
receive the measurements or information derived from the measurements from the at least one sensor;
using the measurements or he information, determine a context associated with the user;
apply at least one warning rule to the determined context and the received measurements or information; and
trigger a warning based on applying the plurality of warning rules.

19. The system according to claim 18, wherein the at least one warning rule is further applied to at least one of additional information from a stored profile of the user, a location of the user, or a time associated with the received measurements or information.

20. The system according to claim 18, wherein the at least one processor is configured to receive the measurements or the information using a wireless low energy beacon signal.

21. The system according to claim 18, wherein the at least one sensor comprises a plurality of sensors.

22. The system according to claim 21, wherein the at least one processor is configured to determine the context using a majority vote amongst the plurality of sensors or by selecting a subset of the plurality of sensors associated with a higher accuracy.

23. The system according to claim 18, wherein the at least one sensor is configured to measure at least one of a location of the at least one sensor, a heart rate of the user, a temperature of an environment of the at least one sensor, a humidity of the environment, or a pressure on the at least one sensor.

24. The system according to claim 18, wherein the at least one warning rule comprises at least one of:
a command to trigger a warning when movement is detected above a threshold,
a command to trigger a warning when movement corresponding to standing up is detected,
a command to trigger a warning when movement corresponding to walking or running is detected,
a command to trigger a warning when movement below a threshold over a predetermined time period is detected,
a command to trigger a warning when a change in lying posture over a threshold time period is not detected,
a command to trigger a warning when movement corresponding to walking during a predetermined time of day is detected,
a command to trigger a warning when a location of the user enters one or more predetermined areas during a predetermined time of day, and
a command to trigger a warning when a location of the user leaves one or more predetermined areas.

25. The system according to claim 18, wherein the plurality of warning rules comprise a subset of predetermined rules selected by an administrator of the system.

26. The system according to claim 18, wherein at least one of the plurality of warning rules comprises a new rule generated by an administrator of the system.

27. The system according to claim 18, wherein the at least one processor is further configured to:
receive additional measurements related to the state of at least one sensor;
apply at least one device state rule to the additional measurements; and
trigger a device state warning based on applying the at least one device state rule.

28. The system according to claim 27, wherein the at least one processor is further configured to playback one or more previous contexts associated with the user in response to triggering the device state warning.

29. The system according to claim 18, wherein the at least one sensor includes an image sensor.

30. The system according to claim 29, wherein determining the context further comprises classifying one or more images from the image sensor.

31. The system according to claim 18, wherein the at least one sensor includes an audio sensor.

32. The system according to claim 31, wherein determining the context further comprises classifying audio from the audio sensor.

33. The system according to claim 18, wherein the warning comprises at least one of a graphical user interface (GUI), a tone, a vibration, or activation of a warning light, and wherein the warning is transmitted to a device associated with the user or an administrator of the system,

34. The system according to claim 33, wherein the device comprises at least one of a personal computer, a tablet, a mobile phone, a pager, a monitor, or a smart watch.

35. The system according to claim 33, wherein the device is integral with the at least one sensor.

36. The system according to claim 18, wherein the at least one processor comprises a first processor integral with the at least one sensor and configured to determine the context and derive the information from the measurements a second processor configured to apply the plurality of warning rules to the determined context and the information.

37. A device for processing sensor signals and providing real-time warnings based thereon, comprising:
detecting at least one variable related to a user using at least one sensor;
at least one processor configured to:
receive, from at least one sensor, measurements or information derived from the measurements related to an individual, wherein the measurements were taken substantially continuously;
using the measurements or the information, determine a context associated with the individual;
apply at least one warning rule to the determined context and the received measurements or information; and
trigger a warning based on applying the plurality of warning rules; and
at least one feedback device configured to transmit the warning to a user of the device.

38. The device according to claim 37, wherein the at least one feedback device comprises one or more of a display, a speaker, a haptic vibrator, or a light.

39. A method for providing real-time warnings based on sensor signals, comprising:
detecting at least one variable related to a user using at least one sensor;
receiving, using at least one processor, measurements or information derived from the measurements from the at least one sensor;
using the measurements or the information, determining a context associated with the user;
applying at least one warning rule to the determined context and the received measurements or information; and
triggering a warning based on applying the plurality of warning rules.
